SEQUENCE LISTING (1 sequence)

SEQ ID NO: 1 - protein, 139 aa, Artificial Sequence
  Human BMP-2
MHVASLRAAAPHSFVALWAPLFLLRSALADFSLDNEVHSSFIHRRLRSQERREMQREILSILGLPHRPRPHLQGLHNSAPMFMLDLTNAMAVEEGGGPGGQGFSTPTKAVFSTQGPPLASLQDSHFLTDADMVMSFVNL

(19) United States
(12) Patent Application Publication
(10) Pub. No.: US 2006/0122109 A1
(43) Pub. Date: Jun. 8, 2006

(54) Composition for preventing the formation of new scar comprising bmp-7

(76) Inventors: Yang Je Cho, Seoul (KR); In Sik Lee, Seoul (KR); Jung Hyun Hur, Gyeonggi-do (KR); Bo Young Ahn, Seoul (KR); Won II Yoo, Gyeonggi-do (KR)

Correspondence Address: JONES DAY, 222 EAST 41ST ST, NEW YORK, NY, 10017, US

(21) Appl. No.: 10/534,590

(22) PCT Filed: Jul. 4, 2003
(86) PCT No.: PCT/KR03/01323

§ 371 Date: Oct. 17, 2005

(30) Foreign Application Priority Data
Nov. 8, 2002 (KR) 10-2002-0069178

(51) Int. Cl. A61K 38/17

(52) U.S. Cl. 514/12

(57) ABSTRACT

Composition for preventing the formation of new scar, e.g. myofibroblast, having BMP-7 polypeptide is disclosed. The composition for preventing the formation of scar includes an effective amount of BMP-7 (Bone Morphogenic Protein-7) polypeptide of sequence 1. The effective amount is 50 ng/ml-50 μg/ml or 0.1 ng-1 μg/kg by weight and the scar is a corneal scar.

### TECHNICAL FIELD

The present invention relates to composition containing BMP-7 (Bone Morphogenic Protein-7) for preventing the formation of a new scar, and more particularly to composition containing BMP-7 for preventing the formation of myofibroblast.

### BACKGROUND ART

Tseng et al. reported that the amnion is effective on the removal of scars (J Cell Physoil. 1999 June; 179(3):325-35, IOVS 1998;39:S428). In addition, it was also reported that components of the amnion inhibits the generation of scar and cures the scar while the scar is being removed (Bull Hosp Jt Dis Orthop Inst 1990 Spring; 50(1):27-34).

The amnion which surrounds a fetus at the innermost layer of the placenta is a thin translucent membrane having a thickness of about 70 μm, easily separated from the chorion. The amnion shows no rejection symptoms against transplantation since it is an immunotherapically inactive tissue without a blood vessel. In a histological aspect, the amnion is composed of monolayer amnion cells, arranged with simple cubic cells, thick basement membrane and extracellular matrix without a blood vessel. The basement membrane includes components such as type IV collagen, laminin α5 and β1. The amnion has an anti-inflammation action by adsorbing inflammatory cells and inducing apoptosis of inflammatory cells so that the inflammatory cells are not penetrated into wounded tissue, and acts as a basement membrane, thereby promoting the regeneration of epithelium while healing the wounded tissue. In addition, it is also reported that the amnion shows anti-inflammation action by controlling secretion of EGF (epidermal growth factor) and FGF (fibroblast growth factor) as well as prostaglandin and interleukin which are inflammatory cytokine, and additionally shows anti-cicatrical action together with anti-adhesive action since it controls the growth of fibroblast and the differentiation of myofibroblast by downward control of TGF-β (transforming growth factor-β) transmission system. Davis clinically uses the amnion for skin graft initially in 1940.

As reported by Goodrich in 2000, when a scar is cured with the amnion being attached to a torn skin, the scar is recovered rapidly as much as 1.5 time that a scar to which the amnion is not attached. According to the report of Gris, it is checked that an excision operation portion of the skin cancer and skin tissues damaged by a wound are recovered to their normal states without any scar if the amnion is attached thereon while they are cured (Am J Vet Res. 2000 March; 61(3):326-9).

The amnion, currently used in the most ophthalmic medical treatment, is generally used for curing corneal opacity appearing after operation, though its function is not yet revealed.

The cornea is a transparent anterior ocular tissue playing an important role as a barrier for coping with stimulus introduced from outside. The wound healing of the cornea is a very complicated process, which is shown as a result of differentiation and tissumerization of corneal sub-structure. Differently to other parts of the human body, the cornea wound healing is successive processes of various events controlled by many factors. Though the wound healing requires scar formation and vascularisation in various other parts of the human body, the most essential point of the cornea wound healing is to remove the scar, which is formed as a final result, through successive processes of various factors.

Since De Rotth initially applied the amnion in the ophthalmic field for symblepharon and loss of conjunctiva, it has been reported that the amnion is effective to control adhesion, protect a wound portion, promote epithelialization by controlling apoptosis of the epithelium, preserving normal epithelium character, and decrease scar generation by reducing generation of a new blood vessel and inflammation (Retinal and Eye Resrch, 1999 18(3) 311-356). At present, Kin and Tseng are studying for applying various functions of the amnion to various ophthalmic diseases, so the amnion is recently used for curing recurrent pterygium and various intractable eyeball surface diseases such as intractable keratitis, corneal ulcer, corneal chemical burn, corneal perforation and Stevens-Johnson symptoms in the ophthalmic extraocular field.

However, the research for perfect understanding of various effects and functions of the amnion has been seldom revealed so far. Thus, the actual circumstances are that the amnion is provided from amnion providers, belonging only to pregnant women who are determined to be negative to infection (hepatitis B, hepatitis C, syphilis, human immunodeficiency virus (HIV) through the serologic test without complication, the amnion should obtained by Cesarean section, and the instruments used for obtaining the amnion should be all aseptically-treated ones. However, there still remains possibility that the amnion is infected while being treated. Practically, when the amnions preserved in a refrigerator are applied to the eyes and then bacteriologically examined, bacteria are detected in 10% of the amnions. This result is very serious since the amnion may cause more adverse effects when being substantially applied. However, if it is possible to extract materials for controlling a scar from the amnion, such infection may be prevented. Accordingly, it is required to extract such materials and then apply to the human body.

Bone Morphogenic Protein-7 (BMP-7) is known to be concerned in the bone formation and play an important role in the formation of eyeball and tooth when they come into existence. However, it is also reported that BMP-7 is not generated for an adult (Dev Biol. 1999 Mar. 1;207(1):176-88., Exp Cell Res. 1997 Jan. 10;230(1):28-37).

### DISCLOSURE OF INVENTION

The present invention is designed to solve the problems of the prior art, and therefore an object of the invention is to provide composition for preventing the formation of scar, which is extracted from the amnion.

In order to accomplish the above object, the present invention provides composition for preventing of scar formation comprising an effective amount of BMP-7 (Bone Morphogenic Protein-7) polypeptide.

Preferably, the BMP-7 polypeptide is of sequence ID No.1.

The effective amount of BMP-7 polypeptide is preferably 50 ng/ml to 50 μg/ml in a solution.

A dose of BMP-7 polypeptide is preferably 0.1ng-1 μg/kg by weight, more preferably 1 μg-50 ng/kg by weight. Within this range, BMP-7 shows dose-dependent effects without toxicity, while it shows little effect below the range and is apt to cause foreign body sensation or pain to the eye above the range.

In addition, the composition of the present invention may be used as an agent for preventing fibrosis of various internal organs such as the retina, the liver and the kidneys, and the effect is generally shown by prevention of smad 2 signal due to TGF-β.

The present invention is revealed through experiments as described below in brief.

Inventors of the present invention extract protein from the human amnion, and divide the protein into various sizes. Each fraction is checked to confirm TGF-β preventing ability, and an effective fraction is analyzed using 2-D gel electrophoresis. Points obtained therefrom are analyzed using MALDI TOF.

After the analysis of the largest protein, it is found to be BMP-7, which is also verified by purchasing BMP-7 and relevant antibodies.

By using commercialized BMP-7 (R&D systems 354-BP), HaCat cell (Human skin keratinocyte) and the cornea of an animal are experimented, thereby revealing applicability as a scar preventing agent.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is a photograph for verifying through the western blot that the formation of myofibroblast caused by TGF-β1 is controlled while BMP-7 (200 ng/ml) is processed, in which the lane 1 is to show without TGF-β1 and BMP treatment, the lane 2 is to show only with TGF-β1, the lane 3 is to show only with BMP treatment, and the lane 4 is to show with TGF-β1 and BMP treatment;

FIG. 2 is a photograph showing an SDS-PAGE result of three samples: one having a molecular weight more than 100,000 (the lane 1), another having a molecular weight of 10,000 to 100,000 (the lane 2), and the other having a molecular weight less than 10,000 (the lane 3);

FIG. 3a is a 2-D gel electrophoresis photograph of amnion extracts, and FIG. 3b is a diagram showing an MALDI-TOF result of a stained 2-D spot;

FIG. 4 is a photograph for showing the western immunoblotting result, which verifies that the extracted protein is BMP-7, in which the lane 1 shows recombinant BMP-7 (R&D system), the lane 2 shows amnion extracts (SDS-PAGE), the lane 3 shows recombinant BMP-7 western blot, and the lane 4 shows amnion extract western blot;

FIG. 5 is a photograph for verifying through PCR that the formation of myofibroblast caused by TGF-β1 is inhibited while BMP-7 (200 ng/ml) is treated, in which the lane 1 shows with TGF-β1 treatment, and the lane 2 is shows with TGF-β1+BMP7 treatment;

FIGS. 6a to 6c are photographs showing that the formation of scar is prevented treating BMP-7 after an alkali burn is made to the eye of a rat, in which FIG. 6a shows alkali+BMP-7, FIG. 6b shows alkali treatment, and FIG. 6c shows a normal state of the eye;

FIG. 7 is a graph showing through TNF-α secretion that BMP-7 prevents inflammation;

FIG. 8 shows the cornea having experienced Fibronectin immunostaining for checking the influence of BMP-7 to corneal opacity, which verifies that the BMP-7 treated cornea shows no expression of fibronectin;

FIG. 9 shows the cornea having experienced α-SMA immunostaining for checking the influence of BMP-7 to corneal opacity, which verifies that the BMP-7 treated cornea shows no expression of α-SMA;

FIG. 10 shows the cornea having experienced Collagen IV immunostaining for checking the influence of BMP-7 to corneal opacity, which verifies that the BMP-7 treated cornea shows no expression of Collagen IV;

FIG. 11 shows the cornea having experienced PCNA immunostaining for checking the influence of BMP-7 to corneal opacity, which verifies that the BMP-7 treated cornea shows no expression of PCNA;

FIG. 12 a diagram showing the prevention of myofibroblast differentiation in cornea keratocyte of a rabbit, wherein TGF-1 is treated in the primary cell to check the expression of fibronectin (the lane 2 of A) and α-SMA (the lane 2 of A), and it is verified that such expression of BMP-7 is prevented (the lane 4 of A and the lane 4 of B) through the western blot (A) and ELISA (B); and

FIG. 13 is a diagram showing the prevention of myofibroblast differentiation in Human skin keratinocyte, wherein TGF-1 is treated in the cell level to check the expression of fibronectin (the lane 2 of A) and α-SMA (the lane 2 of A), and it is verified that such expression of BMP-7 is repressed (the lane 4 of A and the lane 4 of B) through the western blot (A) and ELISA (B).

### BEST MODES FOR CARRYING OUT THE INVENTION

Hereinafter, the present invention will be described in detail through embodiments without purpose of limiting the scope of the present invention.

For experiments of the present invention, SD rat (male, 180-200 g, Korea) and New Zealand white rabbit of 2.5 kg are used. A high-qualified reagent is used for cell culture. DMEM/F12 and MEM manufactured by Gibco-BRL (Grand Island, N.Y., USA), a fetal bovine serum manufactured by Hyclone (Logan, UT, USA), and plastic products manufactured by Falcon (Lincoln, N.J., USA) are used. TGF-β1 (a protein composed of polypeptides having two 112 amino acids, about 2 kDa, expressed in Chinese hamster ovary cell line) and BMP-7 (a protein whose source is Human BMP-2 (Met 1-Arg 282) Human BMP-7 (Ser 293-His 431), preserved at −20° C., expressed in Chinese hamster ovary cell line) employs ones manufactured by R&D Systems (Minneapolis, Minn., USA), anti-PCNA antibody (rat Proliferating Cell Nuclear Antigen, 36 kDa, mouse IgG2a) is manufactured by Sigma (Grand Island, N.Y., USA), anti-fibronectin antibody is manufactured by BioHit, and collagen IV and α-SMA antibodies (recognizing N-terminal and having reactivity to human, bovine, chicken, frog, goat, guinea pig, mouse, rabbit, rat, dog, sheep and snake species) is manufactured by Sigma (Grand Island, N.Y., USA). Western ECL kit (inducing reaction with the use of Horseradish Peroxidase (HRP) combined to secondary antibody as Western Blotting (Chemiluminescence Luminol Reagent) is manufactured by Santa Cruz Biotechnology (California, USA), immunostain kit (including primary antibody having reactivity to mouse, rat, rabbit, G. pig species and dyed into a brown color) and ELISA kit are manufactured by Zymed LABoratories Inc. (San Francisco, Calif., USA), and microscope and digital camera are manufactured by Nikon (Japan).

### First Embodiment

### Extracting Protein from the Amnion

The amnion was obtained from a healthy woman delivered of a child by a caesarian operation.

10 g of the amnion was washed three times in a physiological saline solution, and then grinded in a mortar together with 10 ml of PBS.

The obtained liquid by grinding was then centrifuged to remove sediment. Extract solution obtained in this process was then passed through a membrane having a molecular weight of 100,000 (Amicon Inc.). The collected liquid, not passing through the membrane, was mixed with PBS and then passed again through the membrane, so the extract liquid was separated on the basis of the molecular weight of 100,000. The obtained extract liquid having a molecular weight over 100,000 was then separated on the basis of a molecular weight of 10,000 with the use of a membrane having a molecular weight of 10,000.

### Second Embodiment

### Measuring Ability of the Amnion Extract Liquid for Preventing Transformation of Hacat Cells

HaCat Cell Culture

HaCat cells (Human skin keratinocyte) was cultivated in MEM having 10% FBS within a incubator of 5% CO₂, 37° C. At this time, if more than 90% of cells were grown in the dish, the cells are serum-depleted by MEM (Minimum Essential Medium), not including 10% FBS, for 24 hours.

Measurement of Transformation and Inhibitory Ability

HaCat cells, cultivated to have 2×10⁵ cells in a 6-well plate, was treated by TGF-β1 (5 ng/ml) and the amnion extract liquid for each control group and each molecular weight. After the treatment, myofibroblast was induced for 24 hours. An amount of fibronectin generated was measured by ELISA (Table 1).

At this time, anti-fibronectin Ab (Accurate, IMS02-060-02) having a concentration of 10 μg/ml was attached to a 96-well flat bottom plate by using a coating buffer (0.1 M carbonate buffer, pH9.6). And then, after 1% BSA blocking, fibronectin standard and incubated fluid were treated, and color-developed using anti-fibronectin Ab, HRP (Accurate IMS04-060-02), and then its amount is measured.

TABLE 1
Inhibitory Ability of Myofibroblast Formation Amnion
Extract by TGF-β1 for each molecular weight

| | No | Only | TGF less | TGF 10,000 | TGF more |
|---|---|---|---|---|---|
| | TGF | TGF | than 10,000 | to 100,000 | than 100,000 |
| Absorbance | 0.1 | 1.3 | 0.9 | 0.1 | 1.2 |

### Third Embodiment

### 2-D Gel Electrophoresis and MALDI-TOF Analysis of Amnion Extract

Protein Analysis of Extract Liquid

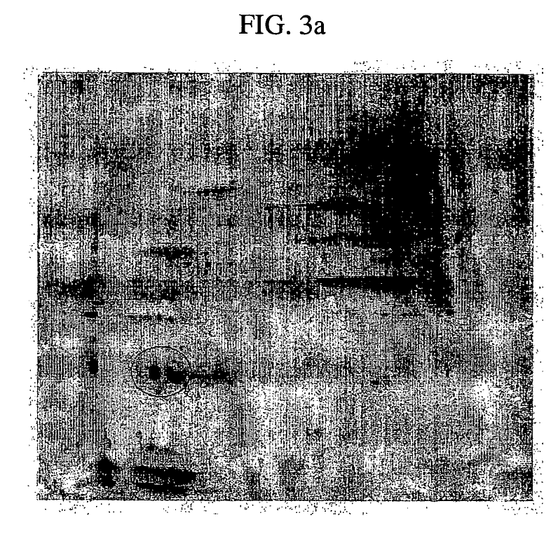

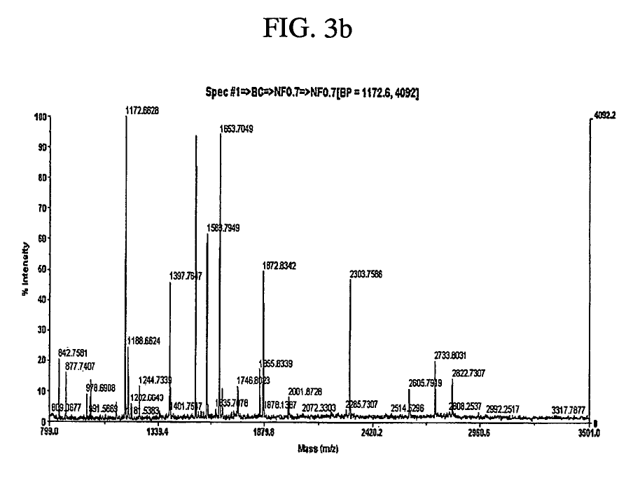

The amnion extract having a molecular weight of 10,000 to 100,000 was made into 1 mg/ml of protein, and then 0.5 ml was obtained from the protein. 1.5 ml of TCA/Acetone was then applied to the protein. Then, precipitate, obtained by centrifugation, was washed by acetone, and then dissolved and boiled in 10 μl of 10% SDS and 2.5% DTE solution. IEF (isoelectric focusing electrophoresis) is conducted thereto with the use of pH 3-10 IPG gel strip (amersham pharmasia biotech), and then it was stained by Coomassie Blue G250 after electrophoresis (see FIG. 3).

Main spots of the stained gel are cut, and entrusted for analyzing protein sequence with the use of ESI-TOF MS/MS using MALDI-TOF and Micromass Q-TOF MS (Australian Proteome Analysis Facility). As a result, the spot was revealed to be BMP-7.

Table 2: Internal Sequence Analysis of Amnion Extract

Sample EG265
Matching protein;
BMP-7 [Homo sapiens]
1 hnsapmfmldlynama
2 fstqgpplaslqd

### Fourth Embodiment

### Checking BMP-7 using Western Immunoblotting

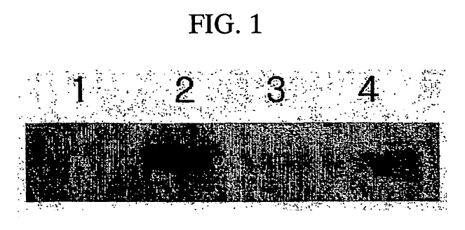

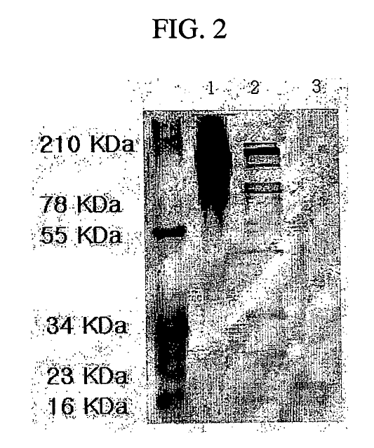

The amnion extract having a molecular weight of 10,000 to 100,000 was made into 1 mg/ml of protein, and then 0.5 ml was obtained from the protein. 1.5 ml of TCA/Acetone was then applied to the protein. Then, precipitate, obtained by centrifugation, was washed by acetone. And then, SDS-PAGE was conducted with the use of 10% Acrylamide gel, and the resulting gel was transferred to a nitrocellulose membrane. Then, western blotting was conducted thereto with the use of BMP-7 monoclonal antibodies, so it was checked that BMP-7 exists in the amnion extract (see FIG. 1).

### Experimental Example

### Test of BMP-7 Effect

Control of HaCat Cell Transformation

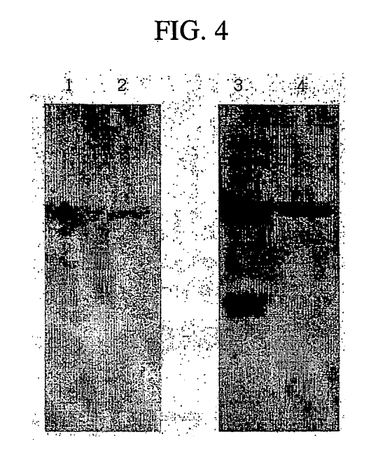

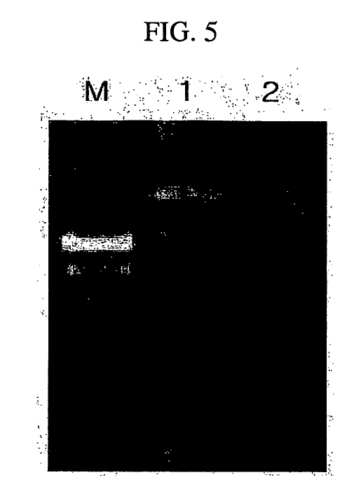

Recombinant BMP-7 (R&D system) expressed from CHO cells was used for checking a HaCat cell transfer inhibitory ability in the same method as the second embodiment. At this time, to check the ability, two methods such as western blotting using fibronectin antibody (see FIG. 4) and PCR using fibronectin gene primer (see FIG. 5) were used.

Test of Scar Formation Inhibition while Rat Cornea having Alkali Burn is recovered

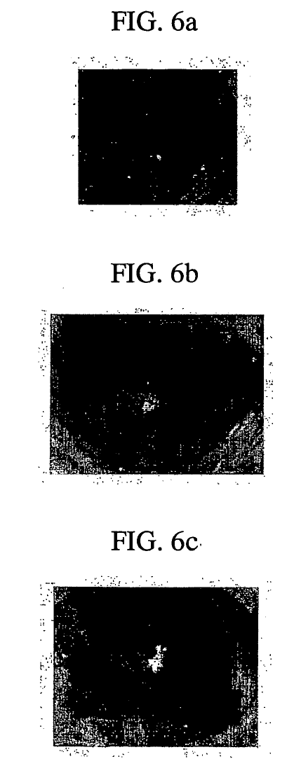

A disk wetted by 1.0 N NaOH was treated to the center of cornea of both eyes of SD rat (male, 180-200 g, Korea) for 60 seconds, and then each 50 μl of medium and BMP-7 (320 ng/ml) was dropped to the left eyeball and the right eyeball respectively. The control group was treated by medium and BMP-7 without NaOH treatment. At this time, the medium and BMP-7 were dropped 4 times a day by a three-hour interval in the day time (10:00 am to 7:00 pm) for 7 days. After that, the eyeballs were photographed after 2 weeks (see FIG. 6).

TNF-α Secretion inhibitory effect using Human Blood

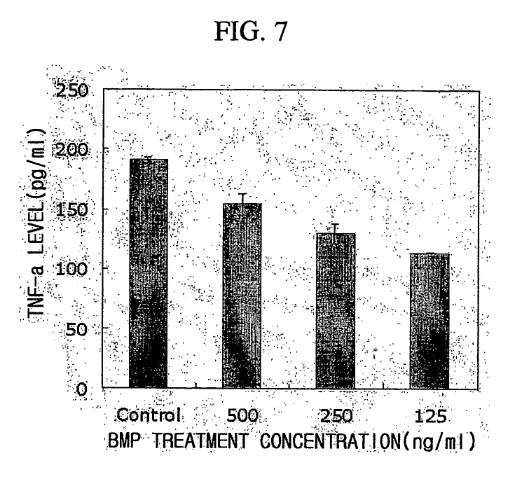

By using a syringe treated by 20 U/ml of Heparin, the same volume of 3% dextran was mixed into a collected blood, and supernatant was separated from the blood after the blood was incubated for about 20 minutes at a normal temperature. Extract obtained by centrifugation makes to be suspended with 20 ml of ice-cold 0.2% NaCl, and then PMN obtained by adding 20 ml of ice-cold 1.6% NaCl was resuspended to 1×¹⁰⁶ cells/ml in PRM1640 including 10% FBS. After that, the liquid was divided to 24 well plate by 1 ml per one well, and E. coli 0127:B8 LPS was added thereto for each well to become 100 ng/ml, and then Saline and BMP-7 are added thereto by various concentrations. After cultivating for 12 hours by 37° C., 5% CO₂, supernatant was collected from each well, and then cytokine secreted using ELISA kit (Human TNF-α quatikine kit, R&D system) was quantitatively analyzed (see FIG. 7).

Immunohistochemical staining of α-SMA, Collagen IV, Fibronectin and PCNA

A disk having a diameter of 25 mm wetted by 1.0 N NaOH was treated to the corneal centers of both eyeballs of Rat, and then washed by 3 ml of saline water. The physiological saline solution as a control group was dropped to the left eye and BMP-7 (320 ng/ml) was dropped to the right eye, 4 times a day (10:00 am to 7:00 pm by a three-hour interval) for 7 days. According to the eyeball ectomy procedure (for each 0 hour, 24 hours, 72 hours, 1 week, 2 weeks, and 3 weeks), the rat was anesthetized by ether and the eyeball was delivered. The delivered eyeball was soaked into paraformaldehyde and fixed at 4° C. for 24 hours, and then serial section was made in a thickness of 4 to 5 μm by using vibratome, and then the immunohistochemical staining was performed.

The sectioned tissue was treated for 3 to 5 minutes in the order of xylene, xylene, 100% EtOH, 90% EtOH, 80% EtOH, and 70% EtOH, then washed three times by phosphate buffered saline (PBS), and then treated by 1% sodium borohydride for 1 hour to remove remained fixing components. As a preprocess for the immunohistochemical staining, the tissue was treated by 3% hydrogen peroxide for 10 minutes, and washed several times by PBS, and then Primary Ab (α-SMA, collagen IV, fibronectin, PCNA) was continuously dropped thereto for reaction so that the tissue was not dried for 60 minutes. After washed by PBS, the tissue was reacted with secondary Ab at a normal temperature for 20 minutes. After that, the tissue was washed again by PBS, then reacted with avidin-biotinylated horseradish peroxidase complex at a normal temperature for 1 hour, then color-developed by a solution of 0.05% diaminobenzidine-tetrahydro-chloride added by 0.01 hydrogen peroxide, then washed by a distilled water, and then performed dehydration and transparency processes with common procedure to make a tissue specimen covered by a glass cover so that it may be observed.

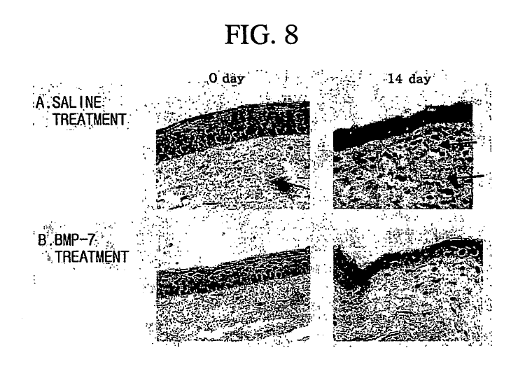

Expression of fibronectin was checked through immunostaining from an alkali-treated cornea of the rat at an initial wound healing, and it was found that the expression was widely spread at a region without BMP-7 treatment after two hours (see FIG. 8).

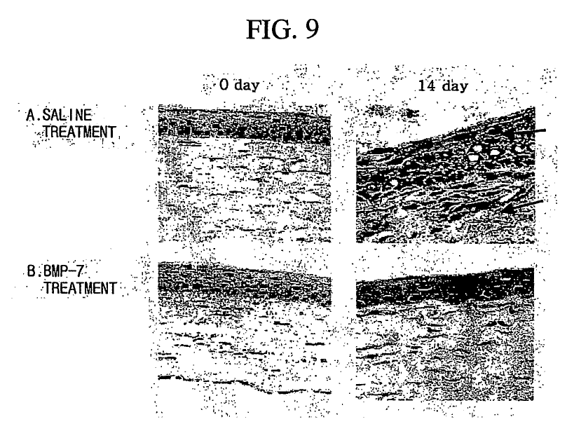

As a result of immunostaining of α-SMA, it was found that the staining was concentrated on cytosol of the cell around the basement membrane in the control group, differently from the BMP-7 treated group. In addition, after 14 days, the control group showed necrosis and tissue degeneration around the basement membrane (see FIG. 9).

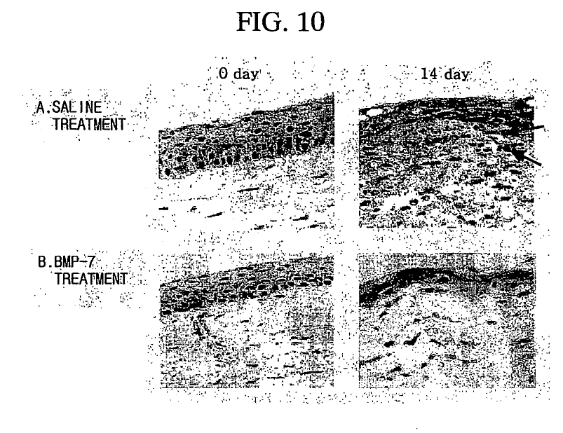

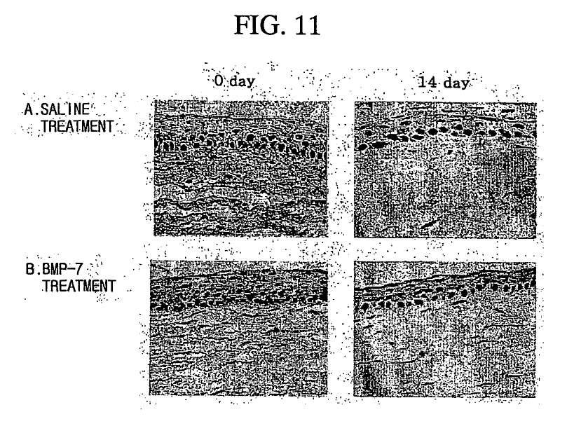

In addition, as a result of collage IV immunostaining which was a main protein forming scar, it was found after 14 days that the staining was more in the control group without BMP-7 treatment (see FIG. 10). On the while, as a result of checking PCNA expression of the alkali-treated cornea of the rat through the immunostaining, it was found that the BMP-7 treated group was better color-developed (see FIG. 11).

Myofibroblast Differentiation Inhibitory Test by TGF-β

i) Rabbit Cornea Keratocyte Primary Culture

An eyeball of New Zealand white rabbit having a weight of 2.5 kg was removed, and then the retina, the choroids and the eye lens are removed. Then, only the cornea layer was dissected and then soaked into Hanks balanced salt solution (HBSS). Collagenase (1 mg/ml) was treated for 12 hours at 37° C. to make it be separated into a single cell. The separated cell was plated to 24 well plate coated by poly-D-lysine. It was used 10% heat-inactivated fetal bovine serum and DMEM/F12 as medium. It was incubated under 37° C. at 5% CO₂ concentration. It was used for experiments 10 to 12 days after culture.

ii) HaCat Cell Culture

HaCat cell was incubated in a tissue culture flask with keeping 37° C., 5% CO₂. MEM having 10% FBS was used as medium, and it is exchanged at every 3 days. If cells were adhered to each other and became submonolayer just before forming monolayer when seen through an inverted microscope, the cells were transferred in the following procedure. The medium in the tissue culture flask were taken out with a pipette, and then the cells were washed by PBS and treated by 0.5% trypsin to take off the cells. The cells, collected by centrifugation in 1,000× g for 3 minutes, were diluted again in culture medium to have 1×10⁵ cells per 1 ml, and then put into a new tissue culture flask. At this time, cell number was measured using a hemocytometer.

iii) Measurement of Differentiation and Differentiation Inhibitory Ability

Corneal cell or HaCat cell, cultivated in 6 well plate to have 1×10⁵ cells, was incubated in MEM for 6 hours, then treated by TGF-β1 (5 ng/ml: added with stock 1 ng/μl-10 μl), and then treated by control group and BMP-7 (200 ng/ml: added with stock 10 ng/μl-40 μl). After the treatment, myofibroblast was induced for 24 hours. An amount of fibronectin and α-SMA generated at this time was measured in western immunoblotting and ELISA.

iv) ELISA

Immunoplates were coated with chicken anti-human fibronectin IgG. This process was conducted overnight at 4° C. while IgG was mixed into 25mM bicarbonate buffer solution to be 1 μg/ml and then put into each well as much as 100 μl. After the coating, the plate was washed three times by PBS. Then, 300 μl of 1% BSA-PBS was put into each well and treated for 1 hour at a normal temperature, and then the plate was washed again by PBS. Specimen, standard solution (human plasma fibronectin) and sample were added to each of the prepared wells as much as 20 μl. The plate was reacted overnight at 4° C., and then washed three times by PBST (0.1% Tween-20 in PBS). And then, detection Ab (Fibronectin, chicken anti-human Conjugated with HRP) 1% PBS solution was respectively added and reacted further for 2 hours at a normal temperature. After the reaction, the plate was washed three times by PBST, and ABTS solution(substrate of peroxidase) was put therein, and then color change was observed. 50 μl/well of 1N H₂SO₄ was put into the well to quit color reaction, and then the change of light absorption was measured by ELISA reader using 405 nm filter.

v) Western Blot

SDS-PAGE is used with modifying a method of Laemmli. A sample was mixed with a sample buffer in which 0.05 M Tris-HCL (pH 6.8), 2% SDS, 5% β-mercaptoethanol, 10% glycerol, and 0.001% bromophenol blue were mixed, and then heated in 100° C. water bath for 10 minutes to denature protein completely. This sample was separated from protein standard marker in stacking gel of 5% acrylamide and running gel of 6% acrylamide. The running buffer, the stacking gel and the running gel, used here, contains 0.1% SDS, while 80 V was kept during stacking, and 130 V was kept during running. The protein standard marker used here was an Invitrogen product, a mixture of myosin (250 kDa), phosphorylase B (148 kDa), BSA (98 kDa), glutamic dehydrogenase (50 kDa), alcohol dehydrogenase (36 kDa), myoglobin red (22 kDa), lysozyme (16 kDa), aprotinin (6 kDa), and insulin B chain (4 kDa).

After SDS-PAGE (6%), gel was shaken in the transfer buffer (192 mM glycine, 25 mM Tris, 20% methanol) for 15 minutes to be in equilibrium. The transfer buffer was filled in Blotting kit, and then assembled in the sequence of cassette, sponge, two sheets of Whatman 3MM paper, nitrocellulose membrane, gel, 2 sheets of Whatman 3MM paper, sponge, and cassette. After that, with setting the gel to cathode and the nitrocellulose membrane to anode, electric current of 300 mA was applied thereto for 2 hours. Nitrocellulose membrane was taken off, and the gel was put into 5% non-fat milk solution and shaken slowly for 30 minutes at a normal temperature, and then reacted overnight at 4° C. by anti-fibronectin Ab washed by PBST and diluted to PBST at the rate of 1:500. After the reaction, the nitrocellulose membrane was washed three times by PBST by 3 minute interval, and then reacted with horseradish peroxidase-conjugated anti-mouse IgG diluted by 5% non-fat milk blocking solution at the rate of 1:5000 for 40 minutes with being slowly shaken. The nitrocellulose membrane was then washed by PBST again by 10 minute interval, and then reacted in ECL solution (Santa Cruze Biotechnology) and exposed X-ray film for detection of signal.

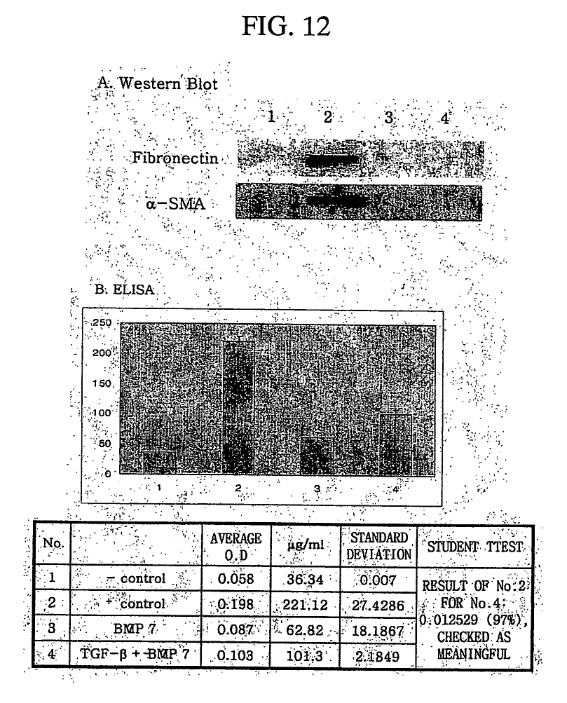

TGF-β was treated to Rabbit cornea keratocyte primary culture cell to check expression of fibronectin and α-SMA, and it was confirmed through the western immunoblotting that BMP-7 might inhibit such expression (see FIG. 12).

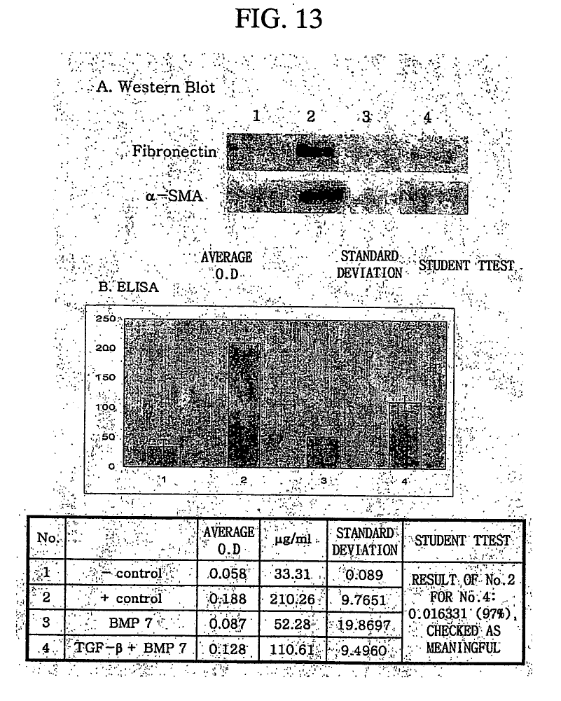

TGF-β was treated to Human HaCat keratocyte primary culture cell to check expression of α-SMA, and it was confirmed through the western immunoblotting that BMP-7 might inhibit such expression (see FIG. 13).

### INDUSTRIAL APPLICABILITY

As described in the embodiments of the present invention, after observing the corneal wound curing process of the rat cornea having alkali burn, it is found that the cornea treated by BMP-7 shows better wound curing without opacity than a control group treated by a saline solution. In addition, in order to investigate the myofibroblast differentiation process in cornea wound curing process, staining fibronectin expressing during the myofibroblast differentiating process and α-smooth muscle actin (α-SMA) which is specific protein of myofibroblast is performed. As a result of staining, it is found that the above two kinds of proteins extensively expressing in the wound curing process are decreased due to the BMP-7 treatment. Moreover, it is also found that stained collagen IV which is an essential protein composing a scar is also decreased while BMP-7 is treated. Generally the inhibition of scar formation may result in the prevention of normal wound curing. But it is found that BMP-7 treatment does not inhibit wound curing from staining of proliferating cell nuclear antigen (PCNA) to observe the effects of BMP-7 on a wound curing velocity.

In addition, by separating cornea of a rabbit, cultivating keratocyte and treating TGF-β, it is induced expression of fibronectin and α-SMA. At this time, it is also found that BMP-7 treatment may inhibit TGF-β activity. Human-derived HaCat cell also shows the same results.

Thus, BMP-7 may be used for inhibiting the formation of scar in the cornea and the skin by inhibiting transformation of myofibroblast, as well as for forming a bone as well known in the art. By using this result, BMP-7 may be as an agent for inhibiting the formation of scar, which is apt to arise during a plastic operation or a laser operation of the cornea.

1. A composition for inhibiting a scar formation comprising an effective amount of BMP-7 (Bone Morphogenic Protein-7) polypeptide of SEQ ID NO:1.

2. The composition of claim 1, wherein the effective amount of BMP-7 is 50 ng/ml-50 μg/ml or 0.1 ng/kg-1 μ/kg.

3. The composition of claim 1, wherein the scar is a corneal scar.

4. The composition of claim 2, wherein the scar is a corneal scar.
